(19) United States
(12) Patent Application Publication
(10) Pub. No.: US 2020/0155125 A1
(43) Pub. Date: May 21, 2020

(54) ORAL SAMPLING APPARATUS AND USES THEREOF

(71) Applicant: The Procter & Gamble Company, Cincinnati, OH (US)

(72) Inventors: Yumeng OUYANG, Beijing (CN); Lei TIAN, Beijing (CN); Jinman LIU, Beijing (CN); Jie ZHANG, Beijing (CN)

(21) Appl. No.: 16/683,794

(22) Filed: Nov. 14, 2019

Related U.S. Application Data
(63) Continuation of application No. PCT/CN2017/088177, filed on Jun. 14, 2017.

(51) Int. Cl. A61B 10/00; A61F 13/38

(52) U.S. Cl. CPC A61B 10/0051; A61B 2010/0006; A61F 13/38

(57) ABSTRACT

Oral sampling apparatus for visually assessing treatment effectiveness of an oral care product in decreasing the amount of bacteria in an oral cavity and method of use thereof.

### FIELD OF THE INVENTION

The invention is directed to an oral sampling apparatus and methods for using the apparatus to allow consumers to visually assess the oral cleanliness and treatment effectiveness of an oral care product.

### BACKGROUND OF THE INVENTION

Demonstrations are effective and inexpensive means to promote and market new products, or new benefits of existing products, to the consumers. In particular, demonstrations can be used to determine a degree of cleanliness of an oral cavity, for example, by measuring the amount of bacteria present in a biological sample (e.g., saliva, plaque) collected from the oral cavity of a subject. When the amount of bacteria is high in the obtained biological sample, the oral cleanliness is considered to be poor, and consequently the cleaning efficacy of the oral care product is considered to be low. Conversely, when the amount of bacteria is low in the obtained biological sample, the oral cleanliness is considered to be good, and the cleaning efficacy of the oral care product is considered to be high.

Regardless of whether the demonstration is live (i.e., with a presenter) or standalone (i.e., self-evaluation), it is desirable that the demonstration articles used are portable to various sites, easy to use (e.g., with minimal instructions required), easy to understand results (e.g., with minimal explanation), and/or occupy minimal display space. Unfortunately, these properties can be difficult to achieve when the demonstrations involve the oral cavity.

Sampling swabs are well-known tools for collecting samples to assess for the presence and/or levels of contaminants (e.g., bacteria) deposited on surfaces of an individual, package or bag. A sampling swab for collecting bio samples and transferring the samples into a test liquid containing luciferase fluorescence to detect the number of bacteria within the test liquid by an ultraviolet color change has been described. See e.g. CN Patent No. CN102634448B (Ningbo) and CN Utility Model No. CN202530081U (Ningbo). Another sampling swab having a sampling head made of plastic nylon for improved collection of epithelial cells from an oral cavity and transferring the obtained sample to a laboratory for measurement has been described. See e.g. CN Utility Model No. CN201949050U (Ningbo). Unfortunately, those sampling swabs are not designed to obtain biological samples from an oral cavity or if they are intended for the oral cavity are not suitable to collect biological samples (e.g., saliva, plaques) related to oral cleanliness. Furthermore, those sampling swabs tend to require heavy and sensitive detection equipment or the like to measure the presence and/or level of bacteria or other biological materials. This equipment is difficult to transport and set-up, which makes these approaches not ideal for use in demonstrations. Furthermore, some of these sampling swabs may require advanced examination techniques by a technical expert, doctor or the like, thus limiting their application.

Accordingly, the need exists for an improved method and oral sampling apparatus for users to visually assess the treatment effectiveness (i.e., oral cleanliness) of an oral care product in an oral cavity that does not require complicated equipment or specialized training.

### SUMMARY OF THE INVENTION

In a first aspect, the present invention is directed to an oral sampling apparatus for the detection of bacteria in an oral cavity, the oral sampling apparatus comprising:

(a) a swab comprising a handle, a sampling head opposing the handle and a stem connecting the handle to the sampling head; and

(b) a tubular cartridge comprising an open end and an opposing closed end, wherein the closed end forms a well storing a dye composition capable of exhibiting a visible color change upon the presence of bacteria;

where at least the sampling head of the swab is releasably received inside the tubular cartridge through the open end;

where the tubular cartridge further comprises a pierceable membrane positioned inside the tubular cartridge between the releasably received sampling head and the well, and where the pierceable membrane retains the dye composition in the well.

Preferably, the dye is free of luciferase, luciferin, solvatochromatic indicators, or combinations thereof.

In a second aspect, the invention provides for a method of assessing the presence of bacteria in an oral cavity, the method comprising the steps of:

(a) providing the oral sampling apparatus, as set forth herein;

(b) releasing the releasably received swab from the tubular cartridge;

(c) obtaining a biological sample of the oral cavity with the sampling head of released swab;

(d) returning the swab with the obtained biological sample into the tubular cartridge;

(e) piercing the pierceable membrane using the sampling head of the returned swab and immersing the sampling head into the dye composition retained in the well; and

(f) visually assessing whether the dye composition of the immersed sampling head exhibits a visible color change as indicative of the presence of bacteria.

In a third aspect, the present invention provides a kit for assessing effectiveness of an oral care product in decreasing the presence of bacteria in an oral cavity comprising at least one oral sampling apparatus, as set forth herein, optionally packaged inside of an opaque bag, and user instructions. Preferably, the bag is a foil bag, preferably a laminate foil bag.

In a fourth aspect, the present invention provides for an oral sampling apparatus having detected bacteria in an oral cavity, the oral sampling apparatus comprising:

a swab comprising a handle, a sampling head opposing the handle and a stem connecting the handle to the sampling head, where the sampling head comprises a biological sample obtained from an oral cavity; and

a tubular cartridge comprising an open end, an opposing closed end and a pierceable membrane therein between;

wherein the swab is irreversibly received inside the tubular cartridge such that the handle is no longer exposed for grasping; and wherein the sampling head having pierced the pierceable membrane and having immersed into a dye composition, where preferably the dye compositions exhibit a color change indicating the presence of bacteria.

It is advantageous that the method and oral sampling apparatus are easy to use and convenient for the users to interpret the results without significant technical assistance. It is potentially advantageous that the method and oral sampling apparatus are easily transportable to various sites to be used in demonstrations to consumers, without the need for additional heavy and sensitive detection equipment. It is also advantageous that the amount of reagents used for testing the bacteria present in a biological sample is limited, which is cost efficient and less leakage risk. It is also advantageous that the structural configuration of the oral sampling apparatus complies with safety requirements for use of the dye (e.g., avoiding leakage) to permit consumer self-evaluation, preferably unsupervised with the oral sampling apparatus. It is also advantageous that the method and oral sampling apparatus are predictive of clinical effectiveness for treatment with the oral care product. It is further advantageous that the method and oral sampling apparatus of the present invention can be used as a quick screening tool for oral care actives.

These and other features of the present invention will become apparent to one skilled in the art upon review of the following detailed description when taken in conjunction with the appended claims.

### BRIEF DESCRIPTION OF THE DRAWINGS

While the specification concludes with claims particularly pointing out and distinctly claiming the invention, it is believed that the invention will be better understood from the following description of the accompanying figures in which like reference numerals identify like elements, and wherein:

FIG. 1 shows a cross-sectional view of an oral sampling apparatus (1) according to an embodiment of the present invention.

FIG. 2 shows a cross-sectional view of a modified embodiment of the oral sampling apparatus (1) of FIG. 1, where further comprising a securing mechanism (14).

FIG. 3 shows a figure with instructions for how to use the oral sampling apparatus (1) to test for treatment effectiveness of oral care products.

FIG. 4 shows a drawing of the oral cavity with labels to indicate different positions to swab and obtain biological samples.

### DETAILED DESCRIPTION OF THE INVENTION

It is to be understood that the scope of the claims is not limited to the specific devices, apparatuses, methods, conditions or parameters described and/or shown herein, and that the terminology used herein is for the purpose of describing particular embodiments by way of example only and is not intended to be limiting to the claimed invention. Also, as used in the specification including the appended claims, the singular forms “a”, “an”, and “the” include the plural.

As used herein, the term “about” when placed before a numerical value “X” refers to an interval extending from 10% of X, preferably 5% of X, and even more preferably to an interval extending from 2% of X.

As used herein, any of the terms “comprising”, “having”, “containing”, and “including” means that other steps, ingredients, elements, etc. which do not adversely affect the end result can be added. Each of these terms encompasses the terms “consisting of” and “consisting essentially of”. Unless otherwise specifically stated, the elements and/or equipment herein are believed to be widely available from multiple suppliers and sources around the world.

As used herein, the term “consumers” is meant to include the customers who purchase the product, users of the product, or the store owners or managers who decide whether to stock their shelves with the product.

As used herein, the term “oral care product” is meant a product, which in the ordinary course of usage, is not intentionally swallowed for purposes of systemic administration of particular therapeutic agents, but is rather retained in the oral cavity for a time sufficient to contact dental surfaces (i.e., tooth) and/or oral tissues for treatment. The oral care product may be in various forms—compositional or an implement—including toothpaste, dentifrice, tooth gel, subgingival gel, mouth rinse, mousse, foam, dental floss, toothbrush, water pick, denture cleaning composition, or denture adhesive. The term “oral care product” may also include treatment regimens where these aforementioned compositions may be applied to teeth via an implement, such as a toothbrush or the like. The term “oral care product” may also include individual oral care actives.

By “tubular” used herein with respect of cartridge, it means that the cartridge is elongated having a longitudinal axis, which can have any cross-section shape, e.g. circle, regular or irregular polygon (e.g., triangle, square, rectangle, etc.), oval, petal, heart, and combinations thereof.

### Oral Sampling Swab

It has importantly been found that the oral sampling apparatus of the present invention is an effective communication tool that allows consumers to visually assess the treatment effectiveness (i.e., oral cleanliness) of an oral care product to an oral cavity. It works by obtaining a biological sample (e.g., saliva, plaque) from a consumer's oral cavity and providing a visible color change to confirm the presence and/or level of bacteria in the obtained biological sample to reflect the degree of oral cleanliness of the oral cavity. Preferably, biological samples are taken pre- and post-product usage to show a correlation of the presence and/or level of bacteria in the oral cavity (i.e., oral cleanliness) to the product's efficacy. The oral sampling apparatus advantageously works without the need for heavy or sensitive or expensive detection equipment such that it can be used broadly by consumers, particularly for self-evaluation. Preferably the oral sampling apparatus also provides for a quick “a-ha” moment by the consumers of a product's performance without requiring a professional background and/or a lot of explanation, yet still be clinically meaningful enough to be accepted by professionals (e.g., dentists).

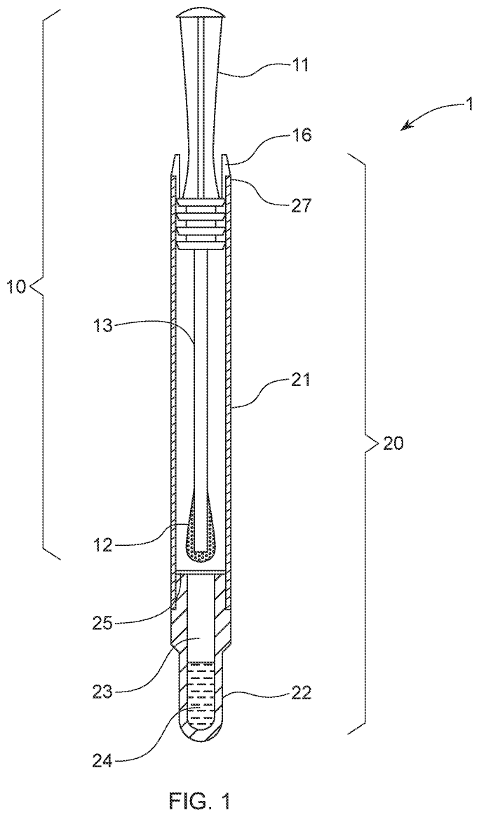

The oral sampling apparatus can be of any dimensions so long as it is small enough to be easily transportable and conveniently storable. For example, referring to the embodiment illustrated in FIG. 1, the length×diameter dimensions of the oral sampling apparatus (1) can be 10.5 cm×1 cm. Alternative dimensions are possible so long as the oral sampling apparatus (1) is not too short for convenient collection of the biological samples, preferably from the posterior region of the oral cavity. The smaller size is advantageous for transport, particular air mail, for delivery of the oral sampling apparatus directly to consumers' home where the demonstration involves self-evaluation. The oral sampling apparatus can be made of any materials (e.g., plastic) which can be molded or shaped, while still being durable enough to be transported without breaking, and generally cost effective. Preferably, the materials are hard plastics such as polycarbonate, polyethylene (“PE”), polypropylene (“PP”), polyvinylchloride (“PVC”) or the like. The external surface of the oral sampling apparatus (1) may have an optional mark, such as a product logo, linking the oral sampling apparatus (1) to the product to be demonstrated. Other marks, such as a pattern, a character, a logo, a word, a letter, a claim, a slogan, a picture, a photo, or combinations thereof, can be used to make the oral sampling apparatus (1) appear less clinical and more appealing to the consumers.

With continued reference to FIG. 1, the oral sampling apparatus (1) comprises a swab (10) and a tubular cartridge (20). The swab (10) comprises a handle (11), a sampling head (12) opposing the handle (11), and a stem (13) connecting the handle (11) to the sampling head (12). The swab (10) has an overall length from 6 cm to 24 cm, preferably from 12 cm to 20 cm, and more preferably from 15 cm to 18 cm. The inventors have found that the overall length of the swab (10) has an impact on ease with which consumers can use the oral sampling apparatus (1). For example, if the overall length is too long then the consumers have difficulty inserting the swab (10) into the oral cavity to obtain biological samples. However, if the overall length of the swab (10) is too short then it will be difficult to reach the posterior regions of the oral cavity. With the recited overall length, the consumers can handle the swab (10) to easily fit the swab (10) inside the oral cavity to obtain the biological samples from all areas of the oral cavity.

The sampling head (12) is designed to collect biological samples (e.g., saliva, plaque) obtained from the oral cavity. The oral cavity is selected from the gum, teeth, interface between the gum and teeth, tongue, oral mucosa, or combinations thereof. The sampling head (12) comprises an absorbent material, and preferably the absorbent material is made of a cellulosic fabric comprising of cotton, linen, rayon, or flax, or combinations thereof. Preferably, the absorbent material of the sampling head (12) is cotton since it is more suitable for collecting biological samples (e.g., saliva, plaque) from the oral cavity and provides an acceptable feel in the oral cavity to the consumers. Preferably the absorbent material is white in color (when dry) as the white color helps to highlight on color changes from the dye composition. Furthermore, the cotton containing sampling head (12) is preferred since it tends to not easily release the biological samples (e.g., saliva, plaque) once collected from the oral cavity. The sampling head (12) has a maximum diameter from 0.35 cm to 1.0 cm, preferably from 0.45 cm to 0.80 cm. Herein the maximum diameter means the longest diameter of the cross-section of the sampling head (12) orthogonal to a longitudinal axis (not shown) of the swab (10). The inventors have found that the maximum diameter of the sampling head (12) is important to the optimized performance of this demo tool. For example, if the maximum diameter is too small then the biological samples (e.g., saliva, plaque) collected may not be enough to cause the visible color change. However, if the maximum diameter of the sampling head (12) is too large then it causes discomfort to the consumers when inserted into the oral cavity. With the recited maximum diameter range, the sampling head (12) can obtain enough biological sample (e.g., saliva, plaque) to allow for the color change reaction visible to the naked eye without causing discomfort to the consumers.

Still referring to FIG. 1, the tubular cartridge (20) comprises an open end (21) and an opposing closed end (22). The tubular cartridge (20) has an overall length from 4 cm to 18 cm, preferably from 12 cm to 15 cm, and more preferably from 12.5 cm to 14 cm. The closed end (22) forms a well (23) for storing a dye composition (24) capable of exhibiting a visible color change upon the presence of living bacteria in the obtained biological sample when the sampling head (12) is positioned inside the well (23) and abuts the closed end (22) of the tubular cartridge (20).

The open end (21) and the closed end (22) can be manufactured integrally (as a single unitary piece) or manufactured separately as two or more pieces and thereafter connected with each other by any suitable connecting means such as gluing, slipping with friction screw-in, or snap-lock. The open end (21) and the closed end (22) may have the same or different inner diameters. For example, the open end (21) may have an inner diameter from 0.80 cm to 1.5 cm, preferably from 1.0 cm to 1.2 cm. The closed end (22) may have an inner diameter from 0.40 cm to 1.2 cm, preferably from 0.60 cm to 1.0 cm. The open end (21) and the closed end (22) can be made of the same or different materials. For example, the open end (21) or the closed end (22) can be made of hard plastics such as polycarbonate, polyethylene (“PE”), polypropylene (“PP”), polyvinylchloride (“PVC”) or the like.

The sampling head (12) is connected to the stem (13) at one end of the stem (13). In one example, the sampling head (12) is comprised of a rigid member with an absorbent material (e.g., cotton) disposed on the exterior surface of the rigid member. This rigid member may be simply an extension of the stem (13). The rigid member can be made from plastic. The rigid member (of the sampling head (12)), in one example, may be cylindrical. And the terminal end of the rigid member (of the sampling head (12)) may be made by simply cutting the rigid member orthogonally relative to the longitudinal axis of the swab (10). The absorbent material is disposed on the rigid member so that the disposed absorbent material extends beyond the terminal end of the rigid member. The distance between the terminal end of the rigid member of the sampling head (12) and the portion of the absorbent material that extends beyond this terminal end is a distance from 0.5 mm to 5 mm, preferably from 0.8 mm to 3 mm, as measured along a longitudinal axis of the swab (10). The inventors have found that this distance is important to the optimized performance of this demo tool. For example, if this distance is too small then the sampling head (12) may cause discomfort to the consumers when inserted into the oral cavity for sampling. However, if the distance is too large then it is difficult for the sampling head (12) to pierce the pierceable membrane (25). With the recited distance values, the sampling head (12) can pierce the membrane with minimum force without deforming the overall sampling head (12).

Preferably, the maximum diameter of the sampling head (12) is equal to or slightly smaller than (e.g. less than 1 mm, or less than 0.5 mm) the inner diameter of the closed end (22) of the tubular cartridge (20). When the sampling head (12) is immersed into the dye (24), the absorbent material portion of the sampling head (12) will swell upon absorption, and thus to some extent, stick to the inner wall of the closed end (22). Therefore, it is difficult for the swab (10) to be removed once it has been inserted into the well (23) of the tubular cartridge (20).

Specifically, at least the sampling head (12) of the swab (10) is releasably received inside the tubular cartridge (20) through the open end (21). Preferably, the sampling head (12) and the stem (13) of the swab (10) are releasably received inside the tubular cartridge (20) through the open end (21). The tubular cartridge (20) further comprises a pierceable membrane (25) positioned inside the tubular cartridge (20) between the releasably received sampling head (12) and the well (23) to form a penetrable barrier for retaining the dye (24) in the well (23) between the pierceable membrane (25) and the closed end (22). There is a longitudinal axis which is through the center point of the tubular cartridge, and preferably the membrane (25) is in a plane orthogonal to the longitudinal axis. Preferably, the pierceable membrane (25) can be any membrane which is water-proof and airtight. Preferably, the pierceable membrane (25) is a foil, preferably an aluminum foil. In one example, the foil is placed from 20 mm to 36 mm, preferably from 26 mm to 32 mm from the inner bottom of the closed end (22) along the longitudinal axis of the tubular cartridge (20).

Preferably, the swab (10) further comprises an edge engaging element (16) attached to the handle (11), where the edge engaging element (16) is configured to engage the edge (27) of the open end (21) of the tubular cartridge (20), so as to prevent the swab (10) from piercing the pierceable membrane (25) before its intended use. Herein the term “attached to” means one element is connected to another element directly or indirectly. Preferably, the edge engaging element (16) is squeezable (between fingers) so that it can be disengaged from the edge (27) of the open end (21) of the tubular cartridge (20), after the swab (10) is inserted back after having collected a biological sample and piercing the pierceable membrane (25).

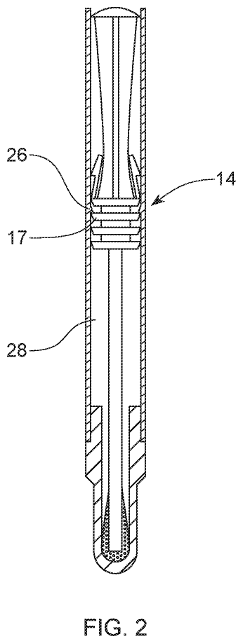

FIG. 2 shows a cross-sectional view of a modified embodiment of the oral sampling apparatus (1) of FIG. 1, further comprising a securing mechanism (14). Features that are in common with those shown in FIG. 1 are designated with the same reference numerals. Referring to FIG. 2, in an alternative embodiment, the oral sampling apparatus (1) may further comprises a securing mechanism (14) for securing, preferably irreversibly securing, the swab (10) to the tubular cartridge (20) upon the sampling head (12) piercing the pierceable membrane, and preferably upon the sampling head (12) abutting the closed end (22). Preferably, the securing mechanism (14) comprises: an inserting element (17) attached to the swab (10); and a locking element (26) protruding from an inner wall (28) of the tubular cartridge (20), such that the insertion of the inserting element (17) through the locking element (26) irreversibly secures the swab (10) to the tubular cartridge (20). The securing mechanism (14) is configured easy in but hard way out which requires less force to push in and more force to pull out. In one example, the inserting element (17) is one or more disk-like protrusions, extending radially out, having about the same cross-sectional area as the tubular cartridge (20). The locking element (26) (complementing the inserting element (17)) extends from at least two opposing points, preferably circumferentially continuous, from the inner wall of the tubular cartridge (20), preferably located at about the middle of the tubular cartridge (20) (so the handle (11) cannot be easily grasped by the user as have been at least partially, if not completely, received inside the tubular cartridge (20)). In this example, the locking element (26) has a downward angled surface allowing the disk-like protrusion to gradually elastically deform when moving past this angled surface (as the swab (10) is received inside the tubular cartridge (20)). However, after the disk-like protrusion of the inserting element (17) moves past this angled surface, the locking element (26) also has a horizontal surface (relative the longitudinal axis of the tubular cartridge (20)) that adjoins to the angled surface. The horizontal surface does not permit the removal of the disk-like protrusion without significant force because of the abrupt deformation that is required to overcome horizontal surface. Consequently, the swab (10) is irreversibly secured to the tubular cartridge (20).

In another example, the locking element (26) can comprise screw threads and the inserting element (17) can be screwed into it, like a “Luer-Lok” mechanism. Other designs of the securing mechanism are also suitable, as long as it would secure, preferably irreversibly secure, the swab (10) within the tubular cartridge (20) once the sampling head (12) is inserted and immersed into the dye composition (24).

After swabbing a biological sample from an oral cavity with the swab (10), the swab (10) is inserted back into the tubular cartridge (20). Upon disengaging the edge engaging element (16) from the edge (27) of the open end (21) of the tubular cartridge (20), the swab (10) is inserted deeper into the tubular cartridge (20) to further pierce the pierceable membrane (25) so as to immerse the sampling head (12) into the dye composition (24).

Preferably, the length of the swab (10) is equal to or smaller than the inner length (i.e. the length from the edge (27) to the bottom of the closed end (22)) of the tubular cartridge (20). In such a way, the swab (10) is irreversibly received inside the tubular cartridge (20) such that the handle (11) is no longer exposed for grasping upon the sampling head (12) piercing the pierceable membrane (25) and preferably abutting the closed end (22).

The sampling head (12) preferably abuts the closed end (22) of the tubular cartridge (20) and is immersed into the dye composition (24). The inventors find that many consumers are confused as where to check for the visible color change when the sampling head (12) does not abut the closed end (22) of the tubular cartridge (20). For example, a cause for confusion is that the visible color change in the dye composition (24) that pools at the closed end (22) of the tubular cartridge (20) may be different from the visible color change in the sampling head (12). Therefore, with the arrangement of the sampling head (12) abutting the closed end (22) of the tubular cartridge (20), the observed visible color change should be the same because the sampling head (12) is immersed in the dye composition (24). This arrangement tends to minimize or avoid confusion to the consumers.

At least a portion of the tubular cartridge (20) is translucent or transparent, preferably transparent, so long as it is convenient for the consumers to view the visible color change reaction between the obtained biological sample and the dye composition (24). Preferably, the closed end (22) of the tubular cartridge (20) is translucent or transparent, preferably transparent, since that is where the dye composition (24) is held.

The closed end (22) of the tubular cartridge (20) forms a well (23) to maintain the dye composition (24). The dye composition (24) is configured to exhibit a visible color change upon reacting with bacteria contained in the obtained biological samples from the oral cavity. Preferably, the dye contained in the dye composition is selected from: 7-Hydroxy-3H-phenoxazin-3-one 10-oxide (“resazurin”); (3-(4,5-dimethylthiazol-2-yl)-5-(3-carboxymethoxyphenyl)-2-(4-sulfophenyl)-2H-tetrazolium) (“MTS”); (2-(4-Iodophenyl)-3-(4-nitrophenyl)-5-(2,4-disulfophenyl)-2H-tetrazolium) (“WST-1”); (2-(2-methoxy-4-nitrophenyl)-3-(4-nitrophenyl)-5-(2,4-disulfophenyl)-2H-tetrazolium) (“WST-8”); 3-[4,5-dimethylthiazol-2-yl]-2,5-diphenyltetrazolium bromide (“MTT”); 2,3,5-triphenyltetrazolium chloride (“TTC”); 2-[4-iodophenyl]-3-[4-dinitrophenyl]-5-phenyltetrazoliumchloride (“INT”); 2,3-bis [2-methoxy-4-nitro-5-sulfophenyl]-2H-tetrazolium-5-carboxanilide (“XTT”); or a salt thereof. Preferably, the dye is resazurin or MTT. Preferably, the dye composition does not contain luciferase, luciferin, solvatochromatic indicators, or combinations thereof. Alternatively, the dye composition can contain any reagent that causes a visible color change upon contact with the living bacteria, with the proviso that the dye does not include a firefly luciferase. Most of luciferase exhibit color change upon ultraviolet light irradiation, which may need involve additional equipment (e.g., a UV lighter) or step, than directly visual awareness of a color change. The dye composition may also contain solvents or other minors. For example, the solvent of the dye composition is water.

The total volume of the dye composition (24) is from 20 μL to 500 μL, preferably from 50 μL to 350 μL. The concentration of the dye in the dye composition is from 20 μg/mL to 10 mg/mL, preferably from 40 μg/mL to 600 μg/mL. Preferably, the dye is MTT. MTT can be metabolized by bacteria and change the color from light yellow to purple which is easily visible to the naked eye, without the help of any special equipment.

In addition to the dye, the dye composition further comprises additional reagents, such as, for example, phenazine methyl sulfate (“PMS”), phenazine ethyl sulfate (“PES”), or combinations thereof. The combination of the dye with the additional reagent, is believed to show a beneficial effect for accelerating the visible color change, particularly in reaction conditions of a short period of time and/or at room temperature, as compared with the case of not including the reagent. The concentration of the additional reagent is from 0.1 to 1 mM, preferably from 0.1 to 0.5 mM.

### Method of Use

In another aspect, the present invention is directed to a method of assessing the presence of bacteria, preferably living bacteria, in an oral cavity. The method can provide to a consumer a visual demonstration of the treatment effectiveness of an oral care product in decreasing the amount of bacteria. The effectiveness of the treatment correlates to the oral cleanliness.

The method according to the present invention, comprises the steps of:

(a) providing an oral sampling apparatus (1) as set forth above;

(b) releasing the releasably received swab (10) from the tubular cartridge (20);

(c) obtaining a biological sample of the oral cavity with the sampling head (12) of released swab (10);

(d) returning the swab (10) with the obtained biological sample into the tubular cartridge (20);

(e) piercing the pierceable membrane (25) using the sampling head (12) of the returned swab (10) and immersing the sampling head (12) into the dye composition (24) retained in the well (23); and

(f) visually assessing whether the dye composition (24) with the immersed sampling head (12) exhibits a visible color change as indicative of the presence of bacteria.

Preferably, the method, as set forth above, further comprises a step of treating an oral cavity with an oral care product. Preferably, the step (c), as set forth above, obtains a biological sample from at least a posterior region of the oral cavity. Preferably, the step (f), as set forth above, assesses the dye composition (24) visible color change over a period of from immediately to 5 minutes, preferably from 10 seconds to 2 minutes, starting upon immersing the sampling head (12) into the dye composition (24). The oral care product may be a mouth wash, strip, toothbrush, dentifrice or toothpaste, preferably a toothpaste.

The present invention also provides a product comprises an opaque foil bag and an oral sampling apparatus (1) as set forth above packaged inside the foil bag, wherein the foil bag is opaque to prevent light from entering therein. Preferably, the foil bag has an inner length of at most 10 mm longer than the total length of the oral sampling apparatus (1) before its intended use, such that the oral sampling apparatus can be further secured within the package to prevent the sampling head from accidentally release or any potential contamination. Alternatively, two or more oral sampling apparatuses (1) as set forth above can be contained in one foil bag for a user to use before and after treatment by an oral care product, i.e. to assess effectiveness of the oral care product in decreasing the presence of living bacteria in an oral cavity.

The present invention also provides a kit for assessing effectiveness of an oral care product in decreasing the presence of living bacteria in an oral cavity comprising a product or the oral sampling apparatus (1) as set forth above. The kit may optionally include instructions for use of the oral sampling apparatus (1) or product comprising thereof for providing to a consumer an assessment of treatment effectiveness.

### EXAMPLE

In order that the present invention described herein may be more fully understood, the following examples are set forth. It should be understood that these examples are for illustrative purposes only and are not to be construed as limiting this invention in any manner.

### Example 1—Visual Assessment of Treatment Effectiveness of Oral Care Product

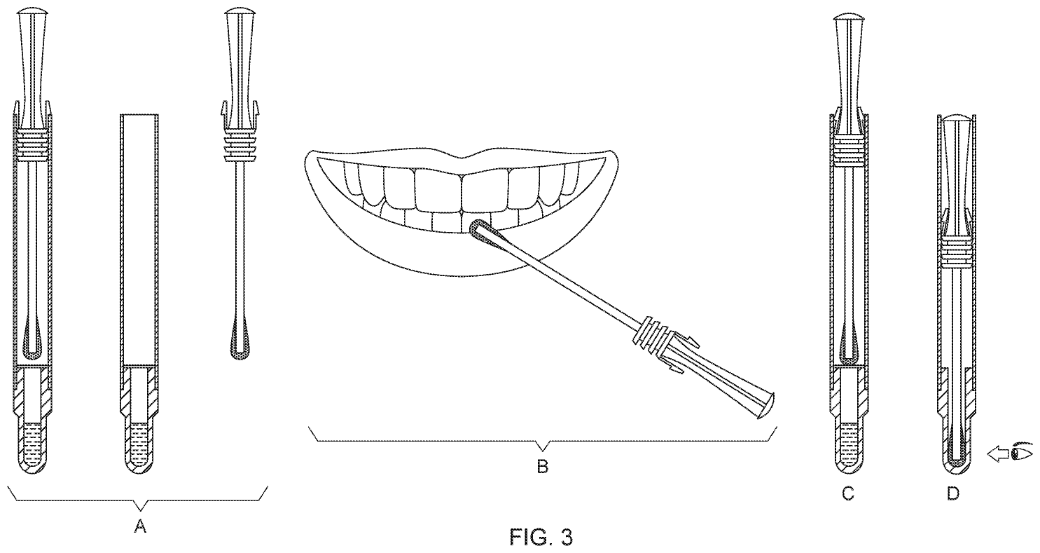

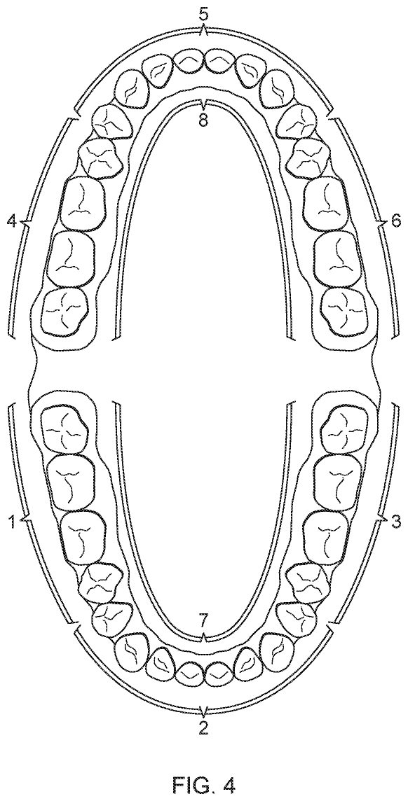

The demonstration tool of the present invention is being utilized to show to consumers the cleaning performance of an oral care product (e.g., Crest® Pro-Health toothpaste—Complete 7 (Procter & Gamble Company, Guangzhou, China)) based on the detection of bacteria in the obtained biological samples by an observed visible color change. The following protocol is used:
1. Study subjects are screened to participate in the test based on the following eligibility criteria: (i) not pregnant or nursing; and (ii) have not brushed their teeth within the last hour.
2. Give the oral sampling apparatus (1) and user instructions (as set forth in FIG. 3) to the subjects. The instructions showed in FIG. 3 may comprise the following steps:
   A. Remove the swab from the tube;
   B. Swab the gum line surface;
   C. Return the swab into the tube and pierce the membrane to immerse the sampling head into the dye; and
   D. Wait for 2 minutes and note the color change for the oral sampling apparatus.
3. Request the subjects to obtain a biological sample from their oral cavity before brushing their teeth with the oral care product by following these instructions:
   (a) grasp the swab handle (11) by the thumb and forefingers horizontally and remove the swab (10) from the tubular cartridge (20);
   (b) swab the gum line surface at the 8 positions as shown in FIG. 4. For positions 1 to 6, swab back and forth twice. For positions 7 and 8, swab back and forth once; and
   (c) return the swab (10) to the tubular cartridge (20) and label the oral sampling apparatus (1) as “Before Brush”.
4. Request subjects to obtain a biological sample from their oral cavity after brushing their teeth with the oral care product by following these instructions:
   (a) brush teeth with Crest® Pro-Health toothpaste—Complete 7; and
   (b) repeat obtaining sampling steps as in 3(a)-(c) and label the oral sampling apparatus (1) as “After Brush”.
5. For both oral sampling swabs (1), piercing the pierceable membrane (25) using the sampling head (12) of the returned swab (10) and immersing the sampling head (12) into the dye composition (24).
6. Leave the sampling heads (12) immersed in the dye composition (24) for 2 minutes.
7. Note the visible color change for both oral sampling swabs (1), wherein a visible color change (e.g., pink color) indicates the presence of bacteria in the obtained biological samples.

Results

5 test subjects are recruited to evaluate the cleaning performance of the oral care product Crest® Pro-Health toothpaste—Complete 7. Subjects are asked to evaluate their oral cavity cleanliness by using the oral sampling swabs “Before Brush” and “After Brush”. The results are discussed below.

For the “Before Brush” biological samples, the oral sampling swabs exhibited a very intense pink color on the sampling head which indicates high amounts of bacteria present in the biological samples. For the “After Brush” biological samples, the oral sampling swabs exhibited a blue (i.e., no color change) or very light pink on the sampling head which indicates very low or low amounts of bacteria present in the biological samples. When the “Before Brush” and “After Brush” oral sampling swabs are compared, a substantially notable visual color change (i.e., blue to intense pink) can be easily visualized by the subjects.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical limitations were expressly written herein.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An oral sampling apparatus (1) for the detection of bacteria in an oral cavity, the oral sampling apparatus (1) comprising:
a swab (10) comprising a handle (11), a sampling head (12) opposing the handle (11) and a stem (13) connecting the handle (11) to the sampling head (12); and
a tubular cartridge (20) comprising an open end (21) and an opposing closed end (22), wherein the closed end (22) forms a well (23) storing a dye composition (24) capable of exhibiting a visible color change upon the presence of bacteria;
wherein at least the sampling head (12) of the swab (10) is releasably received inside the tubular cartridge (20) through the open end (21);
wherein the tubular cartridge (20) further comprises a pierceable membrane (25) positioned inside the tubular cartridge (20) between the releasably received sampling head (12) and the well (23), and wherein the pierceable membrane (25) retains the dye composition (24) in the well (23).

2. The oral sampling apparatus (1) of claim 1, wherein the swab (10) further comprises an edge engaging element (16) attached to the handle (11), said edge engaging element (16) is configured to engage the edge (27) of the open end (21) of the tubular cartridge (20) to prevent the swab (10) from piercing the pierceable membrane (25).

3. The oral sampling apparatus (1) of any of the preceding claims, wherein the pierceable membrane (25) is a foil, preferably an aluminum foil.

4. The oral sampling apparatus (1) of any of the preceding claims, wherein the sampling head (12) comprises an absorbent material, preferably wherein the absorbent material is cotton; and the maximum diameter of the sampling head (12) is from 0.4 cm to 1.2 cm, preferably from 0.6 cm to 1.0 cm, more preferably the maximum diameter of the sampling head (12) is equal to or slightly smaller than an inner diameter of the well (23).

5. The oral sampling apparatus (1) of any of the preceding claims, wherein the length of the swab (10) is equal to or smaller than the inner length of the tubular cartridge (20).

6. The oral sampling apparatus (1) of any of the preceding claims, wherein at least a portion of the tubular cartridge (20) is translucent or transparent, preferably the closed end (22) of the tubular cartridge (20).

7. The oral sampling apparatus (1) of any of the preceding claims, further comprising a securing mechanism (14) for securing, preferably irreversibly securing, the swab (10) to the tubular cartridge (20) upon the sampling head (12) piercing the pierceable membrane, and preferably upon the sampling head (12) abutting the closed end (22).

8. The oral sampling apparatus (1) of claim 7, wherein the securing mechanism (14) comprises: an inserting element (17) attached to the swab (10); and a locking element (26) protruding from an inner wall (28) of the tubular cartridge (20), such that the insertion of the inserting element (17) through the locking element (26) irreversibly securing the swab (10) to the tubular cartridge (20).

9. The oral sampling apparatus (1) of any of the preceding claims, wherein the total volume of the dye composition (24) is from 20 μL to 500 μL, preferably from 50 μL to 350 μL.

10. The oral sampling apparatus (1) of any of the preceding claims, wherein the concentration of the dye composition (24) is from 20 μg/mL to 10 mg/mL, preferably from 40 μg/mL to 600 μg/mL.

11. The oral sampling apparatus (1) of any of the preceding claims, wherein the dye composition (24) comprises a dye selected from the group consisting of: 7-Hydroxy-3H-phenoxazin-3-one 10-oxide; (3-(4,5-dimethylthiazol-2-yl)-5-(3-carboxymethoxyphenyl)-2-(4-sulfophenyl)-2H-tetrazolium); (2-(4-Iodophenyl)-3-(4-nitrophenyl)-5-(2,4-disulfophenyl)-2H-tetrazolium); (2-(2-methoxy-4-nitrophenyl)-3-(4-nitrophenyl)-5-(2,4-disulfophenyl)-2H-tetrazolium); 3-[4,5-dimethylthiazol-2-yl]-2,5-diphenyltetrazolium bromide; 2,3,5-triphenyltetrazolium chloride; 2-[4-iodophenyl]-3-[4-dinitrophenyl]-5-phenyltetrazoliumchloride; 2,3-bis [2-methoxy-4-nitro-5-sulfophenyl]-2H-tetrazolium-5-carboxanilide; or salts thereof; and preferably is 7-Hydroxy-3H-phenoxazin-3-one 10-oxide or 3-[4,5-dimethylthiazol-2-yl]-2,5-diphenyltetrazolium bromide.

12. The oral sampling apparatus (1) of claim 5, wherein the dye composition (24) further comprises phenazine methyl sulfate, phenazine ethyl sulfate, or combinations thereof.

13. The oral sampling apparatus (1) of any of the preceding claims, wherein the oral cavity is selected from gum, teeth, interface between the gum and the teeth, tongue, oral mucosa, or combinations thereof.

14. An oral sampling apparatus (1) having detected bacteria in an oral cavity, the oral sampling apparatus (1) comprising:
a swab (10) comprising a handle (11), a sampling head (12) opposing the handle (11) and a stem (13) connecting the handle (11) to the sampling head (12), wherein the sampling head (12) comprises a biological sample obtained from an oral cavity; and
a tubular cartridge (20) comprising an open end (21), an opposing closed end (22) and a pierceable membrane (25) therein between;
wherein the swab (10) is irreversibly received inside the tubular cartridge (20) such that the handle (11) is no longer exposed for grasping; and wherein the sampling head (12) having pierced the pierceable membrane (25) and having immersed into a dye composition (24).

15. A method of assessing the presence of bacteria in an oral cavity, the method comprising the steps of:
(a) providing the oral sampling apparatus (1) according to any of claims 1 to 14;
(b) releasing the swab (10) from the tubular cartridge (20);
(c) obtaining a biological sample of the oral cavity with the sampling head (12) of the released swab (10);
(d) returning the swab (10) with the obtained biological sample into the tubular cartridge (20);
(e) piercing the pierceable membrane (25) using the sampling head (12) of the returned swab (10) and immersing the sampling head (12) into the dye composition (24) retained in the well (23); and
(f) visually assessing whether the dye composition (24) of the immersed sampling head (12) exhibits a visible color change as indicative of the presence of live bacteria.

16. The method of claim 15, the method further comprising a step of treating an oral cavity with an oral care product.

17. The method of claim 15, wherein the visible color change occurs within a period of time from immediately to 5 minutes, preferably from 10 seconds to 2 minutes.

18. A kit for assessing effectiveness of an oral care product in decreasing the presence of bacteria in an oral cavity, comprising:
a. at least one of the oral sampling apparatus (1) according to any of claims 1 to 13,
b. optionally an opaque bag for containing the oral sampling apparatus, and
c. a user instruction.
